(19) United States
(12) Patent Application Publication
(10) Pub. No.: US 2020/0038160 A1
(43) Pub. Date: Feb. 6, 2020

(54) RETAINING ARC FOR ANCHORING MOTION SENSORS AND METHOD FOR MANUFACTURING SAME

(71) Applicant: FORSTGARTEN INTERNATIONAL HOLDING GMBH, St. Gallen (CH)

(72) Inventors: Frank HORNUNG, Geroda (DE); Stefan KALTENBACH, Rebstein (CH); Stephan WEIHE, Mühlheim an der Ruhr (DE)

(21) Appl. No.: 16/339,289

(22) PCT Filed: Oct. 4, 2017
(86) PCT No.: PCT/EP2017/075252

§ 371 Date: Apr. 3, 2019

(30) Foreign Application Priority Data
Oct. 4, 2016 (DE) 10 2016 012 087.8

(51) Int. Cl. A61C 19/045; A61C 13/34; A61B 90/00; B33Y 50/00; B33Y 80/00

(52) U.S. Cl. CPC A61C 19/045; A61C 13/34; A61B 90/39; A61B 2090/3958; B33Y 80/00; A61B 2090/3912; A61B 2090/3945; B33Y 50/00

(57) ABSTRACT

The invention relates to a device for the reversibly detachable attachment to teeth of an upper jaw or lower jaw, comprising: concave surface regions, each designed for contacting a lateral, in particular buccal, surface region of a tooth to produce a respective positive engagement, the occlusal surfaces of all teeth remaining free so that mastication is not impeded.

### FIELD OF THE INVENTION

The present invention relates to a device for reversibly releasable attachment to teeth of an upper jaw or lower jaw, allowing the patient to perform an unimpeded chewing movement which, for example, can be monitored, recorded and analyzed by further elements mounted on the device. A corresponding production method is also made available.

### PRIOR ART

Devices for anchoring measurement devices are known to a person skilled in the art:

DE202014102968 U1 describes a coupling tray for temporarily fixing mandibular measurement sensors to the lower jaw of a vertebrate, configured as a substantially rigid, box-like shaped part, in particular a shaped plastic part, for introducing mandibular attachment compound for attachment to the front teeth of the lower jaw, said shaped part having, on its top face, a slide surface configured to facilitate sliding of the front teeth of the upper jaw, and with markings which are configured and arranged in such a way as to be detectable by a 3D surface scanner with a detection range smaller than the longitudinal extent of the bow (see FIGS. 14a and 14b). In these and other known techniques, parts of the coupling tray lie in such a way that they partially or even completely cover the masticatory surfaces.

U.S. Pat. No. 7,457,443 B2 describes a method and a device for complex elimination of detection errors, in order to be able to measure and monitor the position of the jaws with greater precision (see FIGS. 15a and 15b). The markers, which are attached to the jaw, are in particular configured to be better identified. Here too, however, the coupling tray, the attachment, covers the occlusion.

DE102009027356 A1 relates to an imaging system for generating a 3D data record (see FIGS. 16a and 16b) of at least part of a jaw by means of an image-generating unit for three-dimensional measurement of an object located within an examination volume, characterized in that a measurement apparatus is present which, during a recording or a series of recordings of the image-generating unit, generates at least one signal (S) which corresponds to the position of the upper jaw (37) and lower jaw relative to each other at least in one direction of mobility, and the images generated by the image-generating unit (11) are assigned to the at least one signal (S). An occlusion tray is shown, with the aid of which the attachment is carried out using an impression compound.

In particular, in the prior art, no retaining devices have been used which allow a natural chewing movement to be recorded, monitored and/or analyzed.

The problem is that, on account of the occlusion being concealed, the terminal occlusion, which is extremely important from the biomechanical point of view, can no longer be attained.

All of the devices according to the prior art have one or more of the following disadvantages:

a) they completely or partially conceal the occlusal surfaces and do not permit a direct terminal occlusion,
b) they require either adhesive or mechanically fixed connections directly on the teeth and are not easily removable afterward,
c) they do not provide the required precision and reproducibility of the position measurement and movement measurement, because of undesired relative movements between dental arch and retaining device.

The object of the invention is to provide a stable attachment device which is easy to fit in place and easy to remove and which permits an unimpeded terminal occlusion. The main aim is to leave the occlusal surfaces on the dental arch free. The attachment is therefore a non-occlusal attachment that can be attached to the dental arch and thus to the jaw of a test subject or patient.

It is a further object of the invention to ensure that the retaining moment against displacement of the retaining arch relative to the dental arch is much higher than was hitherto possible with elastic impression compounds.

### SUMMARY OF THE INVENTION

According to the invention, the object is achieved by a device and a production method according to the independent claims. Preferred illustrative embodiments are set forth in the dependent claims.

According to one embodiment of the present invention, a device is provided for reversibly releasable attachment to teeth of an upper jaw or lower jaw, comprising: concave surface regions, each designed for contacting a lateral, in particular buccal, surface region of a (modified) tooth in order to produce in each case a form-fit and/or force-fit engagement, wherein masticatory surfaces of all the teeth remain free such that a chewing movement is not impeded.

The device can be attached repeatedly to the teeth and released from them again. No glue or adhesive is needed in order to attach the device. Releasing the device does not require adhesive bonds to be torn open. After the proposed device has been removed from the teeth, the teeth do not have to undergo further treatment, for example by polishing. If an adhesive bond is used for the attachment between the device and the tooth, then residues of glue or adhesive in most cases have to be removed subsequently, which is not necessary in the present case. The device is attached principally (if not necessarily exclusively, then possibly exclusively) by a form-fit and/or force-fit engagement of the concave surface regions of the device with the lateral (in particular convex) surface regions of the (modified) teeth. A cohesive bond, for example provided by a glue, is advantageously not involved in the attachment. The teeth can be natural or artificial teeth. It is possible (but not essential) for further elements to be mounted on the teeth, in particular on the lateral surface regions of the teeth, for example by affixing cusps in order to increase a convexity of the surface regions of the teeth. The concave surface regions of the device can be configured to complement convex surface regions of the teeth. In this way, an effective form-fit engagement can be obtained, particularly if a sufficient normal force is applied which acts on the concave surface regions of the device in the direction toward the respective surface region of the tooth. Since the masticatory surfaces remain free, the terminal occlusion or the natural occlusion is not prevented or impeded. This has considerable biomechanical advantages.

The concave surface regions can each have, for example, an area extent of between 0.5 mm² and 10 mm². Other values are possible. According to one embodiment of the present invention, an extent of the concave surface region (or of one of several concave surface regions) of the device is between 30% and 80% of an area extent of the entire lateral surface of the tooth with which the relevant concave surface region comes into contact in the attached state of the device. The greater the area extent of the concave surface region in contact with the respective surface region of the tooth, the greater the possible attachment force with which the device is attached to the teeth.

The device can thus be used, for example, as an auxiliary device or retaining device for components of a system for recording and analyzing a chewing movement of the patient.

According to one embodiment, the concave surface regions can exclusively contact the buccal surface regions of the teeth. In other embodiments, they can engage around a tooth stump, for example, such that buccal and also lingual regions of the stump are in contact with certain regions of the device.

The masticatory surfaces of the teeth are those surface regions of the teeth which, during mastication, come into contact with (opposite) tooth surfaces. In this application, the masticatory surfaces are sometimes also designated as occlusal surfaces, since these surfaces constitute the contact regions during a closed state of the teeth, i.e. a bite state. The device can also be configured to leave free a region adjoining the masticatory surfaces horizontally (from the buccal and/or lingual direction), which region may in fact at times be contacted by opposite teeth during a natural chewing movement.

According to one embodiment of the present invention, the device attachable to the upper jaw may, in the state when attached to the upper jaw, lie completely above the bite plane. According to one embodiment of the present invention, the device attachable to the lower jaw may, in the state when applied to the lower jaw, lie completely below the bite plane. It is thus possible to ensure an unimpeded chewing movement.

The device can be configured as an arc, in particular having substantially a horseshoe shape or a U shape. The device can in particular be configured to extend around the outer side of the teeth. The surface regions of the device and the surface regions of the teeth can each be brought into contact by an elastomechanical force and can be maintained in contact by means of the elastomechanical force. For mounting it on the teeth, the device can be bent slightly open or spread apart at the ends of the “U” and can be positioned on the dentition in the spread-apart state, after which the spreading force can be released such that the ends of the device, or the two arms of the device adjoined to these ends, can be brought into contact with the lateral surface regions of the teeth, on account of the elastomechanical force, and can provide form-fit engagement.

The surface regions of the device and the surface regions of the teeth can at least in part have complementary surface shapes. An effective form-fit engagement can thus be provided.

According to one embodiment of the present invention, the surface regions of the device have at least three, in particular four, concave surface regions (in particular in each case for one tooth or stump) of the device, in particular a left anterior, a left posterior, a right anterior and a right posterior surface region. Here, left, right, anterior and posterior refer to regions of the lower jaw or upper jaw, which thus also define corresponding regions of the device in the attached state. If at least three concave surface regions of the device are present which are brought into contact with three corresponding lateral surface regions of three teeth and provide form-fit engagement, it is possible to achieve a stable attachment of the device. The device can have more than four concave surface regions which come into contact with respective lateral surface regions of the teeth and each provide form-fit engagement. The strength of the attachment can thus be further increased. Between the anterior (e.g. the left anterior or the right anterior) and posterior (e.g. the left posterior or the right posterior) surface regions, an intermediate portion of the device can in each case be provided in which there are no contact regions with teeth or in which there are only contact regions without form-fit and/or force-fit engagement. It is thereby possible that the device is anchored with form-fit engagement at defined positions at a certain (minimum) spacing from each other, such that a stable attachment can be achieved. Otherwise, on account of inaccuracies in production of the device, one contact region could lie close to a further contact region, while a more remote contact region, on account of inaccurate production, is not actually in contact with the lateral surface regions of the teeth. In this case, a stable attachment could not be achieved.

A spacing between the anterior and posterior surface regions can in each case be 0.1 to 0.9, in particular 0.2 to 0.6, times a dentition range from front to rear. The greater the spacing, the more effective and the more stable the attachment can be. The surface regions of the device can, for example, be configured and arranged in such a way that they come into contact, for example, with a canine tooth and with the last or next to last molar. Depending on the degree of a convexity of the teeth and on the spacings between the teeth, it is possible to select suitable lateral surface regions of the teeth which may again define the complementary concave surface regions of the device.

One of the surface regions of the device, in particular all of the surface regions of the device, can be concave in two directions extending transversely, in particular perpendicularly, to each other. If the surface regions or the at least one surface region are/is concave in two non-parallel directions, then the form-fit engagement can provide a retaining force in two directions extending transversely in particular perpendicularly, to each other. It is thus possible to achieve a stable attachment. Depending on the anatomy and shape of the teeth, suitably shaped contact regions on the teeth can be determined and selected, as a result of which the complementary surface regions of the device can also be defined in terms of position and shape.

The device can moreover provide a fastening possibility for an element, in particular a marker and/or a signal transmitter and/or a sensor and/or a light source and/or a bow. In this way, the device can be used, for example, to analyze a chewing movement, so that a natural chewing movement can be performed without obstruction. The marking can be an optical marking, the sensor can be a gyro sensor, for example, and markings or markers can in turn be secured on the bow and can be used for analyzing the geometry and/or the movement of the teeth. Moreover, a signal transmitter or receiver, e.g. a radio transmitter and receiver, can be mounted on the device, by way of which, for example, data determined by the sensor can be transmitted to another system component.

The fastening possibility can be configured as a forwardly protruding arm arranged in the region of the incisors. Thus, without impeding a chewing movement, the arm can be routed outward through the lips, so that other elements can be mounted on the device without causing obstruction.

The device can be designed in multiple parts, in particular in two parts, and it can moreover have at least one coupling element, by means of which the several parts of the device can be coupled while the device is attached to the teeth. The coupling of the several parts of the device causes a pressing force to be applied to the surface regions of the device, which pressing force presses these onto the lateral surface regions of the teeth, resulting in the form-fit engagement. For this embodiment, the device can be made of a rigid, substantially non-elastic material. Moreover, a multi-part design can simplify the mounting of the device. Moreover, a pressing force between the concave surface regions of the device and the lateral surface regions of the teeth can be adjusted by a suitable coupling element, for example a screw, such that an attachment force can be adjusted according to a target value.

The device can be designed in one piece.

The device can be elastically deformable, either in part or overall. The device is preferably elastically deformable in a region that is intended to produce the form-fit or force-fit engagement to a tooth. The attachment of the device to the respective tooth or to the respective teeth can be strengthened by an elastic restoring force.

The device can be produced from an elastic material, in particular plastic. Other materials are possible. The device does not necessarily have to be produced from an elastic material. The elastic material can promote or cause an elastic deformability of the device. The elastic restoring forces, which after the deformation will return the device to the original shape, can strengthen the attachment of the device to the respective tooth. The tooth is then preferably the element which prevents an elastically deformed region of the device from readopting the original undeformed shape. The device is preferably provided to be used in the elastic deformation range of the elastic material and preferably not in the range of the plastic deformation, which would bring about a permanent deformation, with the result that there would no longer be any elastic restoring forces. The elastic material can be a thermoplastic.

Devices made of elastic material may favor the one-part design of the device.

According to an embodiment of the present invention, a method is moreover provided for producing a device for reversibly releasable attachment to teeth of an upper jaw or lower jaw, which method comprises: forming concave surface regions, each designed for contacting a lateral, in particular buccal, surface region of a (possibly modified) tooth in order in each case to produce a form-fit and/or force-fit engagement, wherein masticatory surfaces of all the teeth remain free, such that a chewing movement is not impeded.

Features mentioned in connection with the device may likewise be used for the method, and vice versa.

The method can be implemented partly in software and partly in hardware. It is possible in particular to use visualization or CAD software, with the aid of which a shape of the device can be defined. The method can moreover involve making available a 3D model of the teeth, which model comprises the lateral surface regions, of the teeth of the upper jaw or lower jaw, that are to be placed in contact with the device. The 3D model can in particular comprise all the teeth of the upper jaw or lower jaw, and also regions of the gum. The method can moreover comprise modifying the 3D model by enlargement in regions outside the lateral surface regions provided for the contact. These surface regions not provided for the contact can thus be defined by (virtual or real) application of a certain layer thickness. The shape of the device can be defined by formation of the complement of the modified model. The complement formation can be real or virtual.

The shape of the device can moreover be defined starting from a blank (e.g. a U-shaped blank). The blank can have substantially a horseshoe shape or a U shape. During production, spatial regions corresponding to the modified 3D model of the teeth can be cut out (in reality or virtually) from the blank.

The modification of the 3D model can comprise defining a spacing of between 0.1 mm and 2 mm between surface regions of the teeth not provided for contact and surface regions of the device not provided for contact. It is thereby specifically possible, in an intermediate region between the contact regions, to avoid any contacts between regions of the device and lateral surface regions of the teeth, in order to effect an anchoring by form-fit engagement at defined contact regions that are at a certain spacing from each other.

The 3D model can be made available as a real physical object or as an electronic data record. The modification can be performed on the real physical object or by means of visualization software representing the electronic data record. The shape of the device can be defined by taking a physical impression of the modified model or by complement formation or forming a difference between the blank and the modified model in an electronic data processor.

Thus, the method can be performed in part virtually and in part in physical reality. The method can moreover comprise outputting a data record defining the shape of the device to a 3D printer, and producing the device by means of the 3D printer. Other systems for additive manufacture can also be used for production of the device.

### BRIEF DESCRIPTION OF THE FIGURES

The figures show embodiments of the present invention. The invention is not limited to the embodiments illustrated or described.

FIGS. 1a and 1b show a schematic view of dental arch (1) and retaining arch (4) with interface (5) and with the clamping contact points (41.1 . . . 41.4). Right: the non-occlusal arrangement with the occlusal surface (12) remaining free at the top;

FIGS. 2a and 2b show: Left: Dental arch (1) and jaw (2) as virtual 3D model (3) with virtual occlusal surfaces 32 and buccal contact faces 31 (31.1 to 31.4); Right: Tooth surface in the 3D model with contact face (31.1) in the posterior contact region (44). Top: the occlusal surfaces (1.6) and adjacent tooth (1.5). The cross marks the center of gravity of the pressing force that the retaining arch (4) will exert with contact face (41.1) on the dental arch;

FIG. 3 shows a dental arch (1) as a detail with markings for contact elements (41.1) and (41.2), and with an applied spacing layer (46.1). By means of the spacing layer (46), the retaining arch (4) bears on the dental arch only in the regions (43) and (44). This results in a 4-point attachment. The spacing of the bearing regions to the front and rear is the lever arm HA (45) against vertical deflections when the retaining element is loaded;

FIG. 4 shows a retaining arch (4) of printed plastic with high elastic strength and low plastic deformability. The contact faces 41.1 to 41.4 lie in the posterior molar region and in the canine region. In this way, the retaining device (4) is optimally stabilized with respect to the dental arch (1). The instruments are secured at the mechanical interface (5);

FIG. 5 shows a canine tooth with retaining arch in direct contact. The occlusion 12 at the top is unimpeded. The pressing contact of the contact face 41.1 was obtained by the fact that the 3D shape at the contact was taken directly without modification from the 3D model 31.1. Tooth 1.6, root 1.61 and gum 1.62;

FIG. 6 shows a front molar with retaining arch, with a gap and without contact. The occlusion 12 at the top is also unimpeded here. The gap of the contact face 41.7 was obtained by the fact that the 3D shape at the contact was first reworked by a modification of the 3D model 31.7. Only then was the reworked 3D model used to produce the 3D model 40 of the retaining arch. The retaining arch has the spacing (41.7-31.7) at the tooth shown;

FIG. 7 shows a retaining arch (4) in a side view and plan view. With the anterior contact regions (43) and the posterior contact regions (44) and, lying between them, the spacing regions (46) with spacing HA (45). The load introduced at the mechanical interface (5) is transmitted as force to the contact regions (43) and (44);

FIG. 8 shows a retaining arch (4) additionally with receiving devices for sensor elements (61) or markers (62);

FIG. 9 shows a 3D model (3) of the dental arch, with identification of the planned contact regions (31.1 . . . 31.4) on which the retaining arch will bear with contact faces (41.1 . . . 41.4). In the spacing regions lying between them, a spacing (33.1 and 33.2) is deliberately applied and then reworked with the 3D model to the modified 3D model (30). The modified 3D model (30) then controls the calculation of the virtual retaining arch (40) and the production of the real retaining arch (4);

FIG. 10 shows a virtual 3D model of the retaining arch (40) as control data record for the production of the real retaining arch (4), produced by superposing the modified virtual dental arch (30) with a virtual retaining arch preform (70);

FIG. 11 shows a two-part virtual 3D model of the dental arch (3). The one half 3.1 and the second half (3.2) are deliberately displaced relative to each other such that a reduced spacing is obtained between 31.1 and 31.4. This creates an increased elastic deformation of the retaining arch (4) as soon as it is put onto the dental arch (1);

FIG. 12 shows a view of the non-occlusion embodiment of the retaining arch (40). The retaining arch (4) clamps on the dental arch (1) of the lower jaw (2) in the mouth and carries the sensor equipment (6) at the mechanical interface (5). The relative movement of the lower jaw with respect to the upper jaw, i.e. the movement in the mandibular joint, is measured by optical methods;

FIG. 13 shows the prior art with a tray which conceals the occlusal surfaces and prevents terminal occlusion;

FIG. 14 shows the prior art with a marker which permits determination of the position of the lower jaw but impedes the biomechanically important terminal occlusion;

FIG. 15 shows the prior art with a docked marker for measuring, with an impression tray which prevents the contact of the lower jaw with the upper jaw;

FIGS. 16a to 16f shows the production according to the invention of the virtual 3D retaining arch (40) on the basis of 3D data (3) of dental arch (1) and dentition (2), and also spacing means in the region (46) between the clamping contact faces (43) and (44);

FIGS. 17a and 17b show a real retaining arch (4) of plastic, produced in a 3D printer, with retention faces (41) and mechanical interface (5);

FIG. 18 shows a test subject with inserted retaining arch (4) and a measuring device with markers (6) attached at the interface (5).

### DETAILED DESCRIPTION

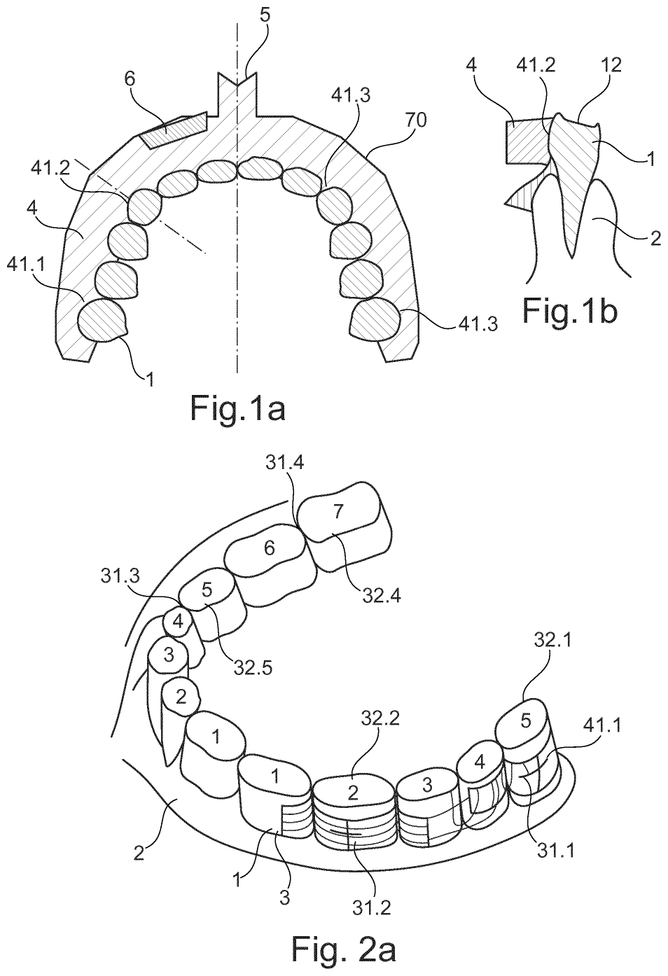

FIGS. 1a and 1b illustrate, e.g. in a plan view, a device 70, 4 for reversibly releasable attachment to teeth 1 of an upper jaw or lower jaw. The device 70, which is also designated as a retaining arch, comprises concave surface regions 41.1, 41.2, 41.3, 41.4 which are in contact with lateral buccal surface regions of certain teeth 1 and provide a form-fit engagement. As is illustrated schematically in a side view in FIG. 1b, masticatory surfaces 12 (also designated as occlusal surfaces) are left free by the device 4 applied laterally from the outside, such that a chewing movement is not impeded.

As can be seen in particular from FIG. 1b, for a lower jaw, the device 4 lies completely below a mastication plane, such that even a lateral movement of opposite teeth (not illustrated in FIG. 1b) during a chewing movement is not impeded by the applied device 4.

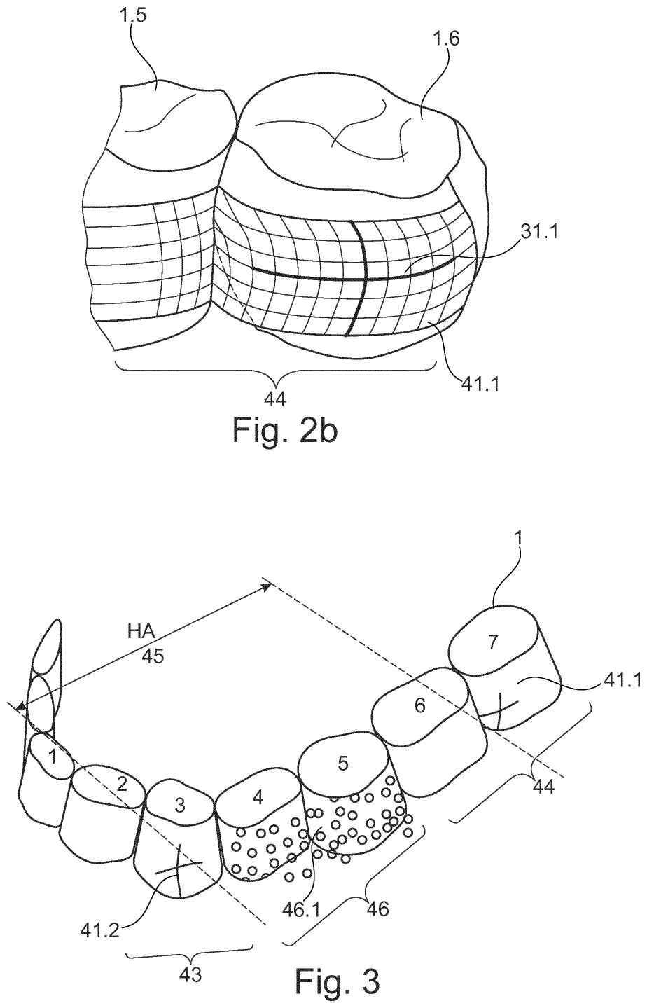

As is illustrated in FIG. 2b, a tooth in the region 44 comprises a surface region 41.1 which, in two almost perpendicular directions (illustrated by solid lines), has a convex shape. In this region, the device 70 or 4 has a complementary concave shape, such that the device in this surface region is formed concavely in two directions almost perpendicular to each other. A form-fit engagement can be improved in this way.

As is moreover illustrated in a schematic perspective view in FIG. 3, in regions 43, 44 the device has concave surface regions which are in contact with convex lateral buccal surface regions 41.1, 41.2 of the teeth. In the intermediate region 46, the device is not in contact with the lateral outer surfaces of the teeth 4 and 5.

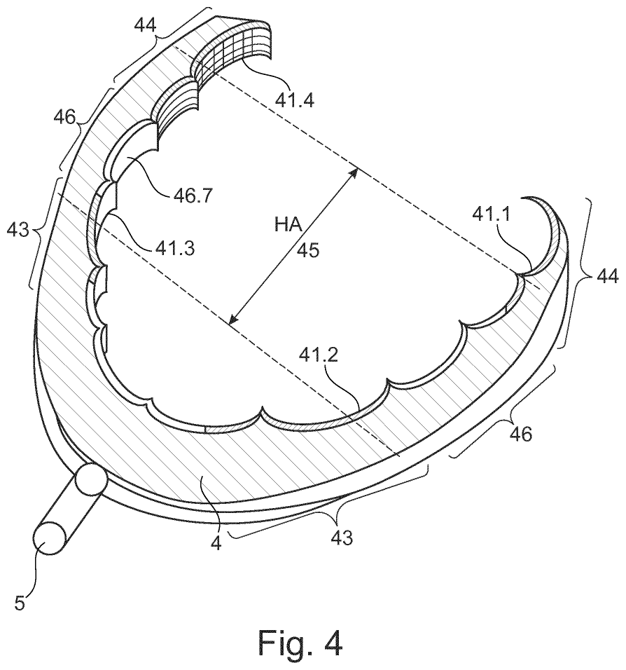

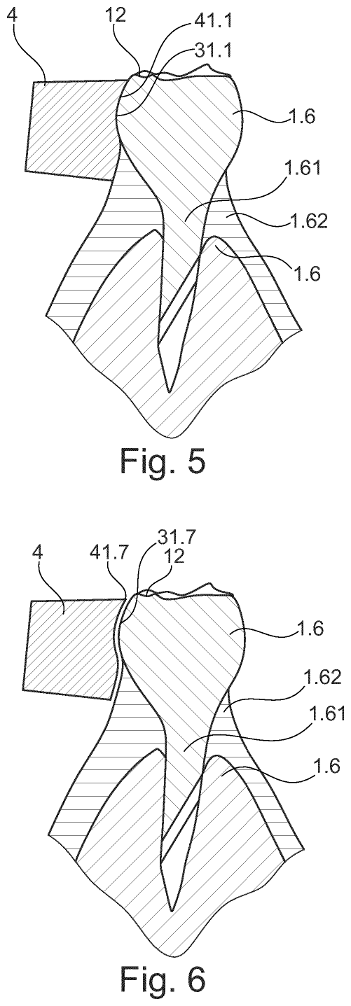

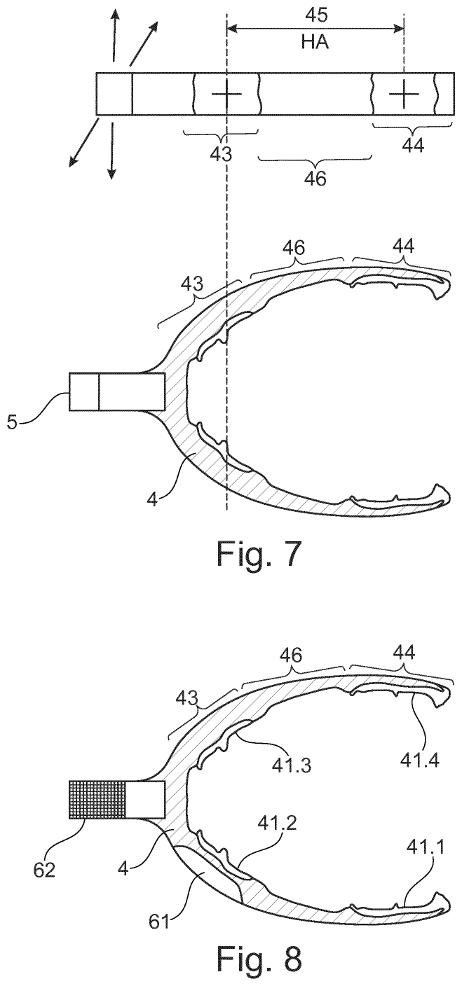

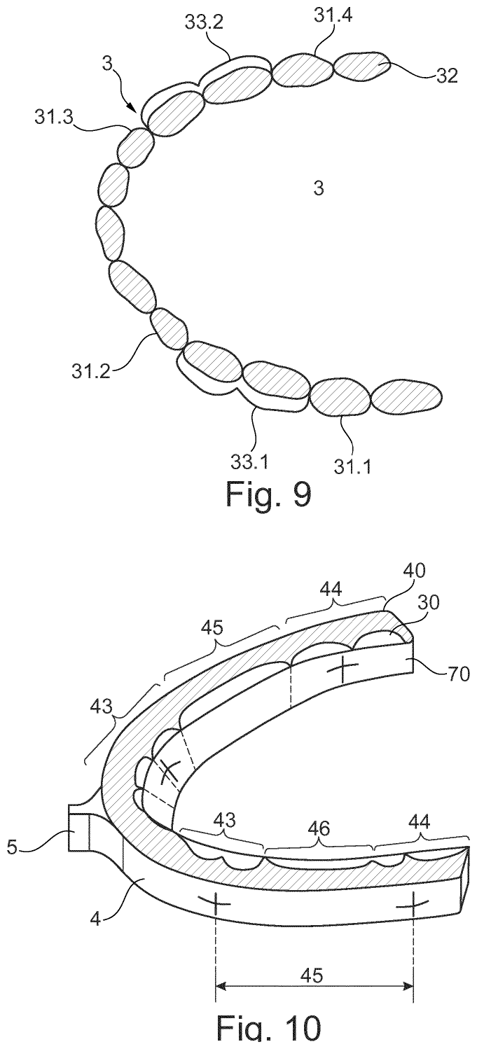

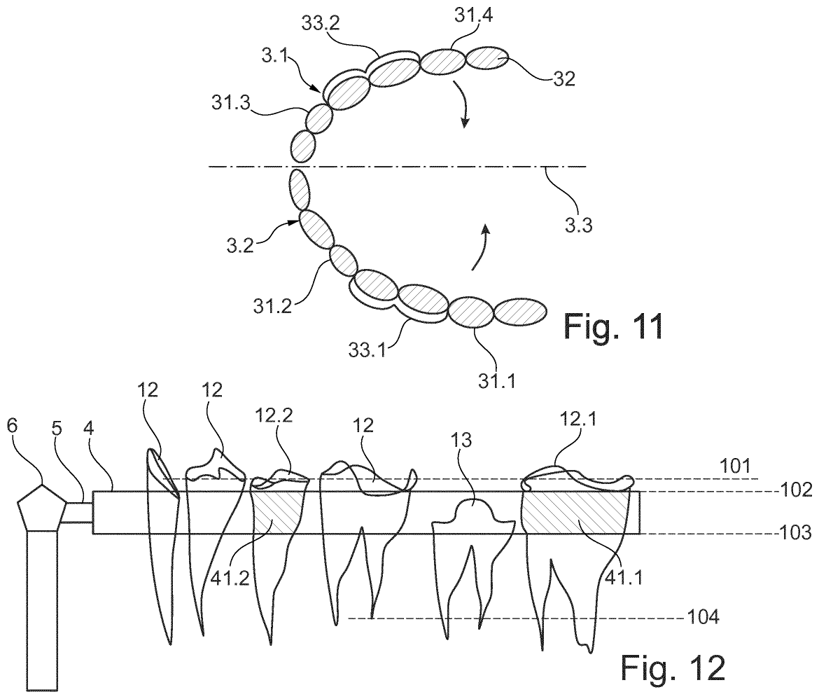

The spacing between anterior and posterior surface regions of the device, which are in contact with lateral surface regions of the teeth, can amount to HA, as is illustrated in FIG. 4 for example. Whereas, in the concave surface regions 41.1, 41.2, 41.3, 41.4, the device 4 is in contact with the convex lateral outer surface 31.1 of the tooth (see FIG. 5), in the intermediate region 46 there is a gap between the lateral outer surface 31.7 of the tooth and the surface 41.7 of the device 4 (see FIG. 6). To obtain this gap in a finished device 4, it is for example possible (virtually or in reality) to apply a certain layer 33.1, 33.2 to those teeth of a 3D model that are not intended to have their outer surfaces in contact with the device, as is illustrated in FIG. 9 for example.

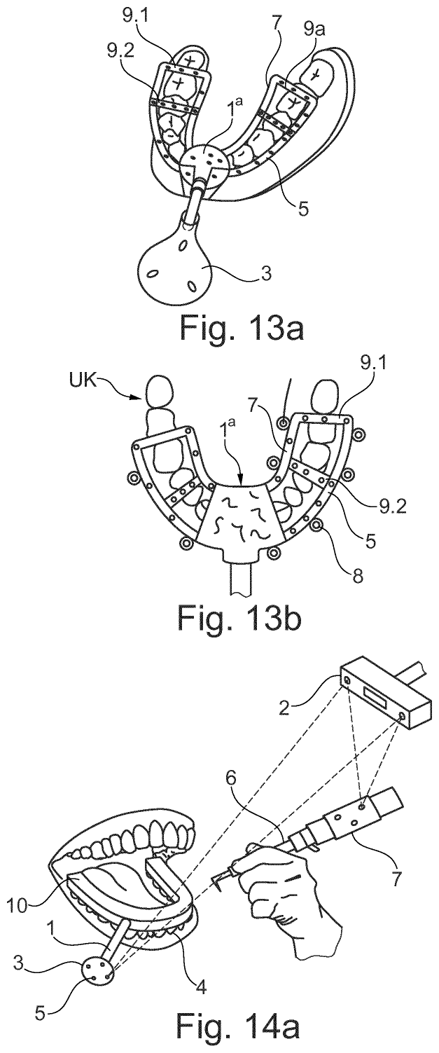

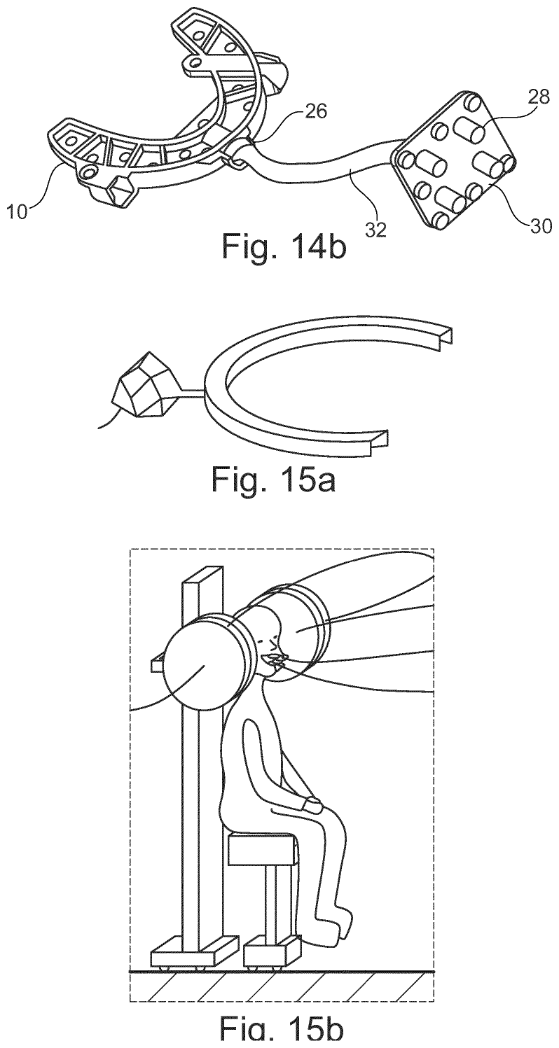

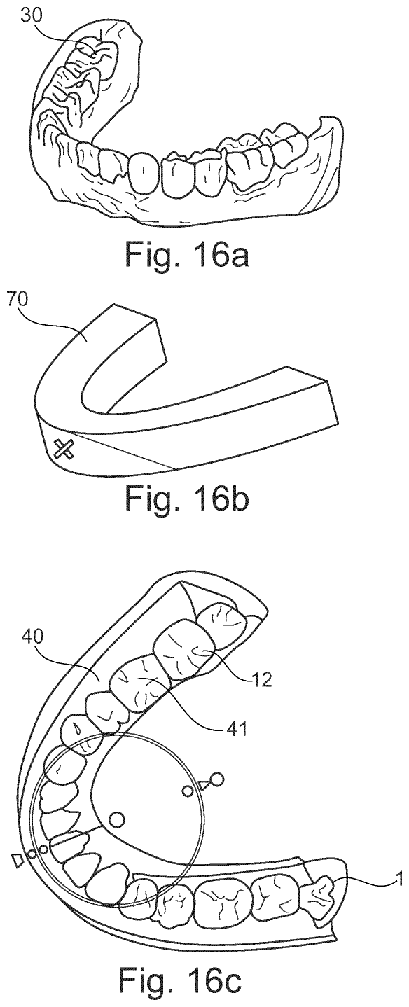

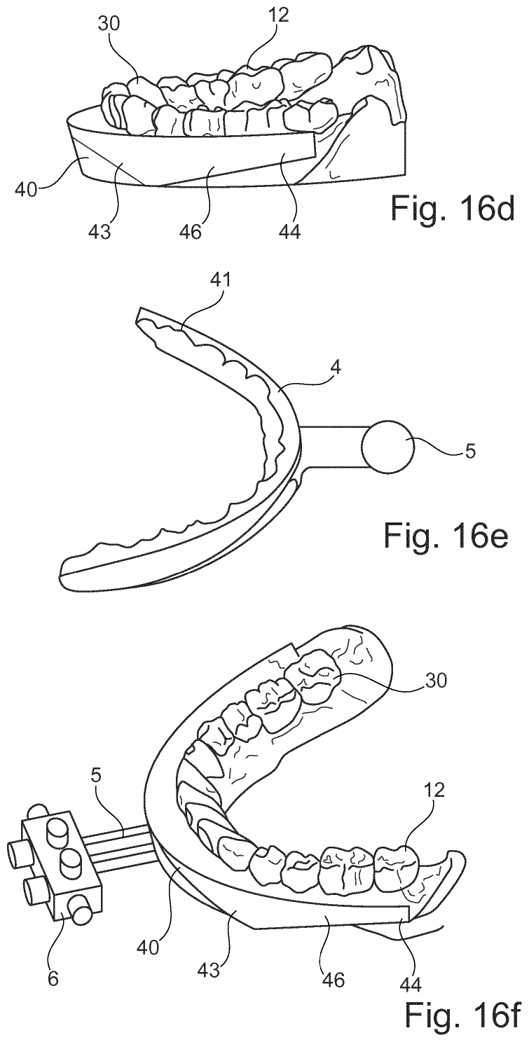

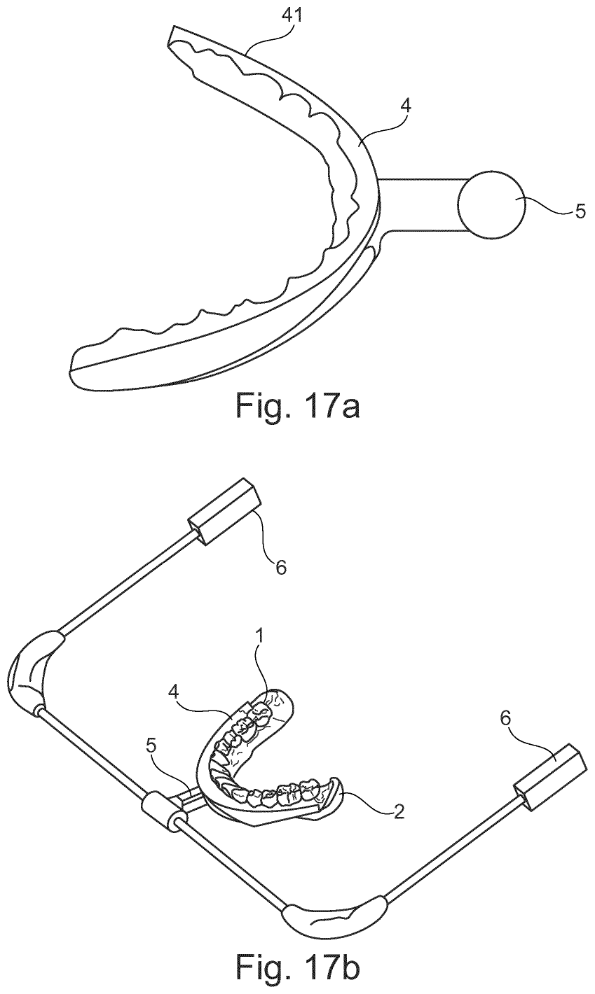

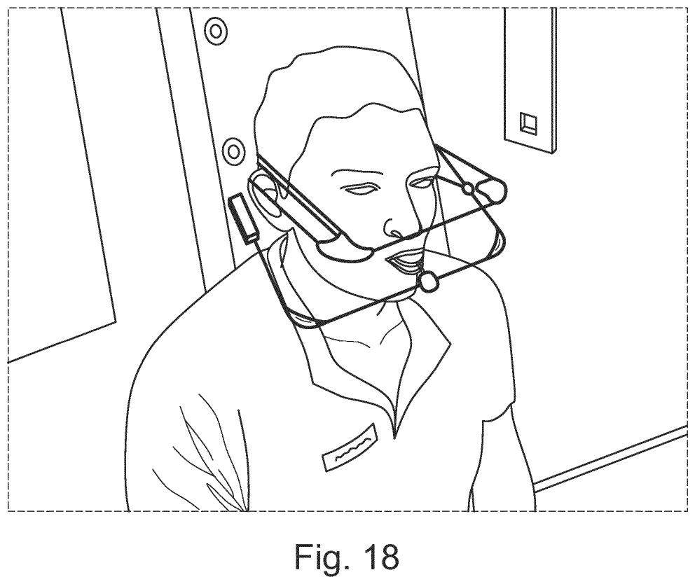

As is schematically illustrated in FIGS. 13a, 13b, 14a, 14b, 15a, various elements can be attached to the device. In this way, it is possible to undertake investigations on the dentition, in particular investigations of a chewing movement. As is illustrated in FIG. 16f, for example, a light source 6 can be attached to the device, or a bow with further elements, as is illustrated in FIG. 17b.

The invention further relates to the reversible and gentle attachment of a retaining device (4) to a dental arch (1), with the aid of non-occlusal lateral contact faces (41.1 . . . 41.4) in a jaw (2), wherein all the occlusal surfaces (12) of the teeth remain free and unimpeded.

The invention furthermore comprises a 3D dental arch (3) produced on the basis of this real model (1), with the objects contained therein such as teeth and implants, and its virtual modification in order to obtain a better press fit for the retaining arch (4).

The particular difficulty is that, if the occlusal surfaces (12) are left free, only the lateral surface regions of the teeth (31), in reality (11) and in particular of the canine teeth and molars, are available for attachment of the retaining arch (4).

With the aid of an imaging process, a 3D model of the dental arch including the gums is established. This is effected either by direct imaging methods or by recording an image of a cast or impression, in particular by X-rays.

The virtual 3D dental arch (3) has a series of occlusal surfaces (32) which have to remain completely free and unimpeded if the meaningful measurements are to be able to be carried out at all. This was not the case with the previously available attachment devices and tray constructions.

The device according to the invention can be made of an elastomechanically loadable retaining arch (4) of flexurally rigid material, with an arrangement of contact faces (41) that exerts a mechanical clamping force and is preferably distributed on at least 3, preferably 4 concave contact surface regions (41.1 to 41.n).

The retaining arch is clamped firmly on the dental arch (1) in a precise position and with a transverse clamping force between the two posterior retaining regions (44), formed of 41.1 and 41.4, and between the two anterior retaining regions (43), formed of 41.2 and 42.3. The spacing between the force centers of the posterior retaining region (44) and anterior retaining region (43) forms the lever arm HA (45).

The positioning precision results from local surface region elements 41 (41.1 . . . 41.n) which are shaped concavely at the contact regions 41 and which are congruent with the convexly shaped buccal and also frontal tooth surfaces 31 (31.1 . . . 31.n).

The occlusal surfaces (1.5 and 1.6) of the dental arch (1) remain deliberately free and permit terminal occlusion even with the retaining arch (4) fitted in place.

By placing the retaining arch (4) onto the corresponding dentition (1), the contact faces (41) can be brought with precise positioning into clamping contact with a dental arch (1). A mechanical interface (5) for attachment of instruments or sensors is located on the retaining arch (4).

Between the contact surface regions (41) having a shape congruent with the dental arch, there are defined spacing surface regions (46, e.g. 46.7) which are deliberately not in contact with the dental arch (1) and instead at a slight distance therefrom.

Between the posterior bearing region (44) and the anterior bearing region (43), there is on both sides a spacing region (46) in which the retaining arch (4) does not bear on the dental arch (3) or bears with only a slight contact force thereon. The centers of gravity of the arrangement of contact faces (41) form an anterior contact group (43) in the canine region and a posterior contact group (44) in the molar region.

The retaining arch (4) according to the invention leaves the occlusal surfaces (12) of the dental arch (1) completely free and limits the contact region to the non-occlusal surfaces (11) of the dental arch (1) and of the mandibular arch (2). The retaining arch extends in the occlusal direction to just under the occlusion plane, and, in the direction of the root, it extends almost to the gum or even reaches the gum in some regions.

In the region of the contact faces (41.1), these bear directly on the tooth (1.1), as per the 3D shaped surface (31.1). However, in the region of the spacing surface (41.7) where no force is supported, there is a gap between the surface (41.7) in the retaining arch and the dental arch with surface (31.7).

The retaining arch (4) is secured on the dental arch (1) by means of an elastomechanical clamping force, via the force-fit and form-fit pressing contact on the contact faces (41).

The clamping between the anterior region (43) and the posterior region (44) generates, over the spacing (45) as long lever arm, a particularly high anchoring moment and very good positioning stability, in particular against transverse and vertical loads via the mechanical interface (5), at which external markers, for example, are mounted.

If, as is the case in the prior art, the clamping arch were to be applied without the spacing regions (46), the transverse clamping would not extend over such a large lever arm HA (45) between the bearing regions (43) and (44), and the retaining moment would be much lower.

In a particular embodiment, the retaining arch comprises a device for the direct application of sensors (61) or transmitters (62) for the purpose of detecting position and movement, in particular of the lower jaw relative to the upper jaw.

A spacing HA acting as lever arm (45), preferably of more than 25 mm, is located between the anterior contact group (43) and posterior contact group (44). By contrast, in previous dental arch impressions, the maximum bearing force lay exactly in the region 46. Retaining arches made of impression material therefore swing about the axis between the regions (46) and are mechanically stable in respect of tilting.

To produce the retaining arch (4) then to be finished, the technology according to the invention does not use the very precise impression-taking and simulation of the buccal 3D surfaces (11.1 . . . 11.n) of the dental arch (1), and instead it uses a very deliberate divergence (33) in the spacing regions (46) between the contact regions (43) and (44).

To produce the retaining arch (4), a virtual 3D dental arch model (3) of the dental arch (1) is used as a starting base. The real dental arch (1) comprises the occlusal surfaces (12), which are imaged in 3D by means of the virtual occlusal surfaces (32).

In the contact surface regions (31.1 . . . 31.4), the 3D model lies as precisely as possible on the 3D data model of the dental arch (1). A precision of between 10 and 100 microns can be achieved with available technology. The spacing regions (33.1 and 33.2) are located between these in the contact regions (31).

At these spacing regions, the virtual 3D model for the dental arch is located farther in the buccal direction, e.g. as a result of an applied layer (33.1 and 33.2), which is applied virtually to the tooth regions lying there. The spacing of the shaped surface 33.2 from the unmodified virtual 3D dental arch surface is ca. 0.1 to 1 mm.

When the retaining arch (4) is placed onto the dental arch (1), the retaining arch is elastically widened to a greater extent in the posterior region (44) than in the anterior region (43). In this way, the required tensioning force is obtained posteriorly, and the retaining arch locks transversely in the plane of the dental arch. The concave shape of the contact faces in the retaining regions (43 and 44) generates the locking of the retaining arch in the two spatial directions perpendicular thereto. The combination of several bearing points (41.1 . . . 41.4) allows the retaining arch to be locked relative to the dental arch in all 6 spatial dimensions (3 degrees of freedom of rotation and 3 degrees of freedom of translation).

The non-occlusal embodiment of the retaining arch (4) entails the arrangement of the arch along a transverse plane through the clamping contact faces 41.1 and 41.2 The occlusal surfaces 11 lie above the retaining arch (4).

In contrast to the prior art, and by virtue of the device according to the invention, the locating or tracking of the movement of the lower jaw in relation to the upper jaw can take place much more precisely and in a manner that is more easily reproducible. This also applies if the device is removed in the meantime and then fitted back in place.

The invention is achieved independently of how the arrangement with anterior bearing regions (43) and posterior bearing regions (44) is produced. An essential feature is the 6D coding by the pressed-on concave contact faces (41) of the retaining arch (4). This guarantees exact and permanently stable attachment of instruments, transmitters, markers or motion sensors, etc.

Illustrative embodiment: Generation of the virtual 3D retaining arch

### INPUT X

Oral SCAN of the dentition with ORAL SCANNER (3)
   Equipment: 3Shape, Sirona, EMS, . . . .
   Format STL
Desktop SCAN of the model
   Equipment: 3Shape, Sirona, CadStar
   Format STL
3D recording of a volume tomograph (Dicom Format) of the model or of the impression tray converted to STL format
   Equipment: CT or DVT
   Format: STL

### INPUT Y

Preformed virtual 3D blank (70) for the retaining arch
Blank of the non-occlusal tray, if appropriate already dimensioned to patient anatomy, dental arch shape for lower jaw.
   Format: STL

### INTERIM RESULT

Widening of the dental arch (3) by the spacing regions (46) with spacing means
Merging to the adapted dental arch model (30)

### OUTPUT

Boolean subtraction quantity as contour of the retaining arch (40)
on the tooth side, with the surfaces of the virtual dental arch (30) adapted for optimal clamping and
top, bottom and outside, with the surfaces of the retaining arch blank (70)

FIGS. 16a, 16b, 16c

Left: adapted 3D dental arch (30) and Center: virtual 3D arch blank (70) matching the jaw geometry (2)

Right: superpositioning of dental arch (30) and arch blank (70)

FIGS. 16d, 16e, 16f

Left: virtual retaining arch (40) with contact regions (43) and (44), adapted dental arch (30), wherein the occlusal surfaces of the dental arch remain completely free

Center: virtual 3D retaining arch (40) as data record for producing the real retaining arch with the tooth-side clamping contact faces (41.1 . . . 41.4)

Right: virtual presentation of dental arch (30) and retaining arch (40) with mechanical interface (5) and marker (6)

FIGS. 17a, 17b

The retaining arch (4) is clamped firmly on the dental arch (1) and bears on the jaw (2) with gum. Measuring equipment (6) is secured at the interface (5). In the image a large, very precise embodiment for optical position determination with the aid of passive markers.

FIG. 18

The retaining arch (4) is clamped on the dental arch (1) of the lower jaw (2) in the mouth and carries the sensor equipment (6) at the mechanical interface (5). The movement of the lower jaw relative to the upper jaw, i.e. the movement in the mandibular joint, is measured by optical methods.

### Further Illustrative Embodiments

Proceeding from a 3D data model (3) of dental arch (1) and lower jaw (2), first of all a layer with a thickness of 0.2 to 0.5 mm is applied virtually to the buccal aspect in the region of the anterior molars. This layer is then merged in the 3D model with the dental arch, resulting in the deliberately modified 3D model of the dental arch (30). The modified virtual 3D dental arch (30) is then intersected with the preform (70) as arch element. This results in the virtual 3D model (40) of the modified retaining arch. By means of a 3D printer, the virtual 3D model (40) is printed out. The edges and peaks are trimmed digitally or on the 3D print. This is followed by sterilization. Thereafter, the retaining arch can be used several times for movement measurements on the patient or in test subjects. If necessary, a new and identical retaining arch can be printed.

In an alternative illustrative embodiment, in the 3D data model of the dental arch (3), the volume of the teeth lying in particular in the spacing region (46) is virtually inflated in 3D, such that the adapted 3D data model of the dental arch (30) there has a spacing gap (33) from the unmodified 3D dental arch (3). The spacing of the modified 3D model (30) is 0.2 to 0.5 mm in the region of the anterior molars (46). The thus modified virtual 3D dental arch (30) is then intersected with the preform (70) as arch element. This results in the virtual 3D model (40) of the modified retaining arch.

In an alternative illustrative embodiment, the 3D model (3) of the dental arch is divided, and one half (3.1) is tilted relative to the other half (3.2) about a small angle or is displaced by a short distance, such that the spacing between the contact faces (31) and (41) is smaller in the posterior contact region (44). The two halves (3.1) and (3.2) are then merged with an adapted 3D model (30) of the dental arch. The spacing of the modified 3D model (30) is 0.0 to 0.5 mm after elastic deformation, in particular in the region of the anterior molars (46). The thus modified virtual 3D dental arch (30) is then intersected with the preform (70) as arch element. This results in the virtual 3D model (40) of the modified retaining arch.

In a particularly preferred embodiment, the virtual 3D dental arch (3) is widened in wide regions by application of virtual cover layers or by inflation of the 3D volumes of the teeth, after which contact surface elements (48.1 . . . 48.4) of exact dimensions with respect to the contact faces (31) are introduced into the retaining arch (4) at the regions (43) and (44) and produce the form-fit clamping contact that transmits force.

With the aid of direct or indirect 3D shaping, the digital 3D model (40) of the retaining arch is converted with precise dimensions into the real retaining arch (4). Examples of available techniques are 3D printing, laser polymerization, 3D milling, etc. The invention also extends to other shaping methods.

### LIST OF REFERENCES AND NUMBERING

Real dental arch
11 real contact faces
1.6 occlusal surface, molar
12 occlusal surfaces of the dental arch
1.61 root
1.62 gum
jaw, lower jaw
virtual 3D dental arch original
first half of 3D dental arch
second half of 3D dental arch
30 virtual modified 3D dental arch with gap at 46
31 contact face at the 3D dental arch, 31.1 . . . 31.4
33 spacing means for generating the spacing regions at 46
4 real retaining arch
40 virtual 3D retaining arch
41 contact faces of the retaining arch in the regions 43 and 44, 41.1 . . . 41.4
41.7 spacing surface in the region 46
46.1 applied layer thickness as spacing means in the virtual 3D model
46.7 3D generation of the spacing surface at 46
43 anterior contact region
44 posterior contact region
45 spacing between the contact regions
46 spacing region
5 mechanical interface
6 sensors and instruments
61 built-in signal transmitter
62 directly mounted marker or LED
70 3D model or virtual retaining arch preform

### MOREOVER, ACCORDING TO THE INVENTION, THE FOLLOWING NUMBERED EMBODIMENTS ARE MADE AVAILABLE

1. A device for anchoring objects in precise position on a dental arch (1) of a test subject for detecting the position and/or movement of the dental arch (1) or of the jaw (2), in particular of the lower jaw relative to the upper jaw, characterized by:
a non-occlusal embodiment as a laterally extending retaining arch (4) with free occlusal surfaces (12) of the dental arch (1) and contact faces (41) on the lateral buccal outer aspects of the teeth,
fixing the retaining arch (4) relative to the dental arch (1) and jaw with gum (2) by elastomechanical forces, which press the contact faces of the retaining arch (41) against the contact faces of the dental arch (11),
coding the position against rotation and translation by the pressing forces acting on the concave contact faces and retentions (41) of the retaining arch (4).
   2. The device as per embodiment 1 for precise spatial anchoring on the dental arch, with
at least 3 bearing points at contact faces (41.1 . . . 41.3) which span a plane,
concave contact faces on the dental arch (11) and on the retaining arch (41) in two dimensions with retentions both in the section line with the spanned plane and also perpendicular to the section line and to the spanned plane.
   3. The device as per embodiment 1 or 2 for precise and repeatedly releasable anchoring of objects on a dental arch of a test subject for identifying the position or movement of the dental arch or of the lower jaw, in particular relative to the upper jaw, characterized in that
two contact regions, anterior (43) and posterior (44), are present,
retaining arch (4) with concave contact faces (41) in the anterior contact region (43),
and with concave contact faces (41) in the posterior contact region (44),
and spacing 45 between posterior and anterior contact region,
lateral mechanical pressing of the contact faces 41 onto the dental arch,
spacing HA (45) in particular greater than 20 mm.
   4. The device as per one of embodiments 1 through 3, in which, between the anterior and posterior contact regions (43) and (44), a spacing region (45) is present in which the retaining arch (4) does not bear on the dental arch (1) or bears only with a slight clamping force thereon.
   5. The device as per one of embodiments 1 through 4, in which, on the retaining arch (4), at least one mechanical interface (5) is present, with fastening device for technical subgroups, in particular sensors (61), signal transmitters, markers (62), etc.
   6. The device as per one of embodiments 1 through 5, in which, on stumps in the dental arch that lie below the occlusion plane, the retaining device covers and engages around the stump.
   7. The device as per one of embodiments 1 through 6, in which, at end-position objects in the dental arch, in particular on isolated molars or implants, the retaining device engages around the stump.
   8. A method for producing one of embodiments 1 through 7, in which
an impression is first of all taken of a real dental arch (1), consisting of a series of objects selected from tooth, crown, filling, implant, stump, gum, and is transferred to a 3D data record of a dental arch (3),
the 3D data record of the dental arch (3) is then virtually processed in a targeted manner in order to generate a reworked 3D data record of the dental arch (30) with in particular two clamping regions 43 and 44,
virtual dental arch (30) and retaining arch preform (70) are then merged, from which the virtual retaining arch (40) results,
a shaping process by 3D CAM is then used in order to produce the real retaining arch (4), in particular 3D printing or 3D milling.
   9. The method as per embodiment 8, in which, between the anterior and posterior contact regions (43) and (44), a spacing region (46) is generated by addition of a thick layer (33) with thickness D of between 0.1 and 2.0 mm on the surface of the real or the virtual dental model in the region (46), and then a 3D model of the retaining arch is generated using the merging of virtual dental arch (3) and thick layer (33).
   10. The method as per embodiment 8 or 9, in which the virtual 3D model of the dental arch is volumetrically inflated partially or completely, such that a modified virtual 3D model of the dental arch (30) is generated and, after generation of a virtual retaining arch (49), a real retaining arch (4) with a spacing gap in the regions (46) is generated by means of 3D shaping, in particular by digital printing.
   11. The method as per one of embodiments 8 through 10, in which, between the anterior and posterior contact regions (43) and (44), a spacing region (46) is generated by addition of a thick layer (33) with thickness D of between 0.1 and 2.0 mm on the surface of the real or the virtual dental model in the region (46), and then a 3D model of the retaining arch is generated using the merging of virtual dental arch (3) and thick layer (33).

1. A device for reversibly releasable attachment to teeth of an upper jaw or lower jaw, comprising:
concave surface regions, each designed for contacting a lateral, in particular buccal, surface region of a tooth in order to produce in each case a form-fit engagement,
wherein masticatory surfaces of all the teeth remain free, such that a chewing movement is not impeded.

2. The device as claimed in claim 1, wherein the device has a horseshoe shape or a U shape.

3. The device as claimed in claim 1, wherein the surface regions of the device and the surface regions of the teeth are each brought into contact and kept in contact by an elastomechanical force.

4. The device as claimed in claim 1, wherein the surface regions of the device and the surface regions of the teeth have at least in part a complementary surface shape.

5. The device as claimed in claim 1, wherein the surface regions of the device have at least three, in particular four, concave surface regions of the device, in particular a left anterior, a left posterior, a right anterior and a right posterior surface region,
wherein, between the anterior and posterior surface regions in each case, an intermediate portion of the device is provided in which there are no contact regions with teeth or in which there are only contact regions without form-fit and/or force-fit engagement.

6. The device as claimed in claim 1, wherein a spacing between the anterior and posterior surface regions is in each case 0.1 to 0.6, in particular 0.2 to 0.4, times a dentition range from front to rear.

7. The device as claimed in claim 1, wherein at least one of the surface regions of the device, in particular all of the surface regions of the device, is/are concave in two directions extending transversely, in particular perpendicularly, to each other.

8. The device as claimed in claim 1, wherein the device moreover provides a fastening possibility for an element, in particular a marker and/or a signal transmitter and/or a sensor and/or a light source and/or a bow.

9. The device as claimed in claim 1, wherein the fastening possibility is configured as a forwardly protruding arm arranged in the region of the incisors.

10. The device as claimed in claim 1, wherein the device is designed in multiple parts, in particular in two parts, and at least one coupling element is moreover provided by means of which the several parts of the device can be coupled while the device is attached to the teeth, wherein the coupling of the several parts of the device causes a pressing force to be applied to the surface regions of the device, which pressing force presses these onto the lateral surface regions of the teeth, resulting in the form-fit engagement.

11. The device as claimed in claim 1, wherein the device is produced from an elastic material, in particular plastic.

12. A method for producing a device for reversibly releasable attachment to teeth of an upper jaw or lower jaw, which method comprises:
forming concave surface regions, each designed for contacting a lateral, in particular buccal, surface region of a (modified) tooth in order in each case to produce a form-fit engagement,
wherein masticatory surfaces of all the teeth remain free, such that a chewing movement is not impeded.

13. The method as claimed in claim 12, wherein the method moreover comprises:
making available a 3D model of the teeth, which model comprises the lateral surface regions, of the teeth of the upper jaw or lower jaw, that are to be placed in contact with the device;
modifying the 3D model by enlargement in regions outside the lateral surface regions provided for the contact;
defining the shape of the device by complement formation of the modified model.

14. The method as claimed in claim 12, wherein defining the shape of the device moreover proceeds from a U-shaped blank.

15. The method as claimed in claim 12, wherein the modification of the 3D model comprises defining a spacing of between 0.1 mm and 2 mm between surface regions of the teeth not provided for contact and surface regions of the device not provided for contact.

16. The method as claimed in claim 12,
wherein the 3D model is made available as a real physical object or as an electronic data record,
wherein the modification is performed on the real physical object or by means of visualization software representing the electronic data record,
wherein the shape of the device is defined by taking a physical impression of the modified model or by complement formation or forming a difference between the blank and the modified model in an electronic data processor.

17. The method as claimed in claim 12, moreover comprising:
outputting a data record defining the shape of the device to a system, which is suitable for additive manufacture, for example to a 3D printer; and
producing the device by means of the system, for example the 3D printer.
